(19) United States
(12) Patent Application Publication
(10) Pub. No.: US 2025/0042903 A1
(43) Pub. Date: Feb. 6, 2025

(54) TRIAZINE INHIBITORS OF CYCLIN-DEPENDENT KINASES

(71) Applicant: H. LEE MOFFITT CANCER CENTER AND RESEARCH INSTITUTE, INC., Tampa, FL (US)

(72) Inventors: Andrii MONASTYRSKYI, Lutz, FL (US); Derek DUCKETT, Land O Lakes, FL (US); Solomon Tadesse ZELEKE, Addis Ababa (ET)

(21) Appl. No.: 18/570,899

(22) PCT Filed: Jun. 17, 2022
(86) PCT No.: PCT/US2022/033947

§ 371 Date: Dec. 15, 2023

Related U.S. Application Data
(60) Provisional application No. 63/211,787, filed on Jun. 17, 2021.

(51) Int. Cl. C07D 487/04; A61K 31/53; A61K 31/5377

(52) U.S. Cl. CPC C07D 487/04; A61K 31/53; A61K 31/5377

(57) ABSTRACT

Disclosed are inhibitors for CDK12/13 and uses thereof. Methods of using the disclosed compounds to treat cancer are also disclosed.

### CROSS-REFERENCE TO RELATED APPLICATIONS

This application claims the benefit of priority to U.S. Provisional Application No. 63/211,787, filed Jun. 17, 2021, the disclosure of which is incorporated herein by reference in its entirety.

### BACKGROUND

There is a continuing need to identify and develop new chemical entities for treating proliferative diseases and conditions, including cancers and myotonic dystrophy. Among the targets for potential antiproliferative compounds under investigation are the group of enzymes known as protein kinases. Cyclin-dependent kinases (CDKs) are a family of protein serine/threonine kinases that are regulated and activated by post-translational modification such as phosphorylation and require interaction with noncatalytic regulatory subunits known as cyclins for their activities (Genome Biol 2014, 15 (6), 122). CDKs control transcription and other critical cellular functions in mammalian cells, including cell proliferation. So far, more than 20 CDKs and 30 cyclins have been reported. Although individual CDKs have multiple cellular roles, CDKs 1-7 have been best characterized in regulating cell cycle progression, whereas CDKs 7-20 have demonstrated roles in transcription and co-transcriptional processes (Development 2013, 140(15), 3079-3093: Malumbres M (2014) cyclin dependent kinases. Genome Biol).

Dysregulation of cell cycle control and transcription are hallmarks of cancer. Genetic alterations, including amplification, overexpression, and mutation of CDKs or their cognate cyclins, contribute to unscheduled proliferation and genomic and chromosomal instability; and aberrant activity of CDKs is a common feature of most cancer types. Therefore, CDKs constitute biomarkers of proliferation and attractive pharmacological targets for the development of anti-cancer drugs (Cancers 2015, 7(1), 179-237; Nat Rev Cancer 2009, 9(3), 153-66). Indeed, three CDK inhibitors targeting the cell cycle regulatory CDKs, CDK4 and CDK6, are approved in hormone receptor-positive metastatic breast cancer. Several inhibitors targeting the transcriptional CDKs, CDK7, CDK8/19, and CDK9, are currently in clinical trials to treat a wide variety of malignancies (Pharmacol Res 2020, 152, 104609; Transcription 2019, 10(2), 118-136).

Cyclin-dependent kinases 12 and 13 (CDK12/13) belong to the group of transcriptional CDKs and, in association with their cognate cyclin, cyclin K, promote transcription elongation through phosphorylation of Ser2 and Ser5 within the YSPTSPS heptad repeats of the carboxy-terminal domain (CTD) of RNA Polymerase II (Pol II) with higher activities when Ser7 is pre-phosphorylated (Cell Div 2012, 7, 12; Nat Commun 2014, 5, 3505). Moreover, unlike other transcriptional CDKs, both CDK12 and CDK13 possess N-terminal arginine-serine-rich domains characteristic of RNA binding and splicing factors. They have been shown to interact with splicing factors directly and regulate splicing and 3′-end processing. Loss of CDK12/13, or cyclin K, impedes both global Pol II processivity and pre-mRNA processing, suggesting redundancy of the two kinases as fundamental regulators of global Pol II processivity and transcription elongation (Mol Cell Biol 2015, 35(6), 928-38; Science Advances 2020, 6(18), eaaz5041).

Upon loss of CDK12 activity, defects in elongation due to premature cleavage and termination as a result of the activation of intronic polyadenylation (IPA) sites that are enriched in longer genes, including DDR genes, were observed (Dubbury S. J et al. (2018) Nature). Thus, the resulting DDR defect (or BRCA-like phenotype) in CDK12 mutant tumors might present an opportunity to develop ‘synthetic-lethal’ therapeutic strategies.

Indeed, ovarian and prostate cancers with functional mutations in CDK12 elicit a BRCAness phenotype with associated hypersensitivity to poly(adenosine diphosphate-ribose) polymerase (PARP) inhibitors and DNA cross-linking agents (Journal of Biological Chemistry 2014, 289(13), 9247-9253; Nucleic Acids Research 2015, 43(5), 2575-2589; Cancer Research 2014, 74(1), 287-97; Cancer Cell 2018, 33(2), 202-216 e6).

In addition, tumors driven by the so-called “undruggable” oncogenes controlled by super-enhancers such as MYC and EWS/FLI highly depend on transcription and DDR gene expression for their rapid replication. Thus, inhibiting the function of CDK12 as both a transcriptional coactivator and a regulator of DNA damage-related proteins is synthetically lethal with MYC overexpression (in neuroblastoma) and EWS/FLI expression (in Ewing sarcoma) (Nature 2011, 474(7353), 609-615; Nat Med 2019, 25(10), 1526; P Natl Acad Sci USA 2012, 109(24), 9545-9550). Furthermore, in human epidermal growth factor receptor (HER) 2-positive breast, gastric, and papillary thyroid cancer, a strong correlation between CDK12 level and high tumor grade exists (BMC Cancer 2010, 10, 539; Theranostics 2020, 10(14), 6201-6215; Journal of Cancer 2020, 11(15), 4308-4315). Studies also propose that CDK12-mediated cell cycle vulnerabilities in cancer exist and are amenable for CDK12 targeted therapy (Nat Commun 2018, 9(1), 1876; EMBO Rep 2019, 20(9), e47592). The role of CDK13 in transcription is less well understood, and CDK1.3 mutations have not been reported in cancer; except for its amplification in hepatocellular carcinoma (HCC), where CDK13 copy number was significantly associated with clinical onset of HCC (PLoS One 2012, 7 (8), e43223). These pre-clinical studies propose that selective CDK12 and/or CDK13 inhibitors have potential use as single agents or in combination therapy in cancer.

In addition, recent studies have expanded the suitability of targeting CDK12 to inflammatory (Science Signaling 2018, 11(541), eaam8216) and myotonic dystrophy type 1 diseases (Science Translational Medicine 2020, 12(541), eaaz2415).

New and potent compounds are needed that inhibit the activity of CDK12 and/or CDK13, particularly for the prevention or treatment of proliferative diseases and conditions, including cancers and myotonic dystrophy. The compositions and methods disclosed herein address these and other needs.

### SUMMARY

In accordance with the purposes of the disclosed materials and methods, as embodied and broadly described herein, the disclosed subject matter, in one aspect, relates to compounds, compositions, and methods of making and using compounds and compositions. In particular, the present disclosure provides novel substituted N-((1H-benzo[d]imidazol-2-yl)methyl)pyrazolo[1,5-a][1,3,5]triazin-4-amine derivatives selected from:

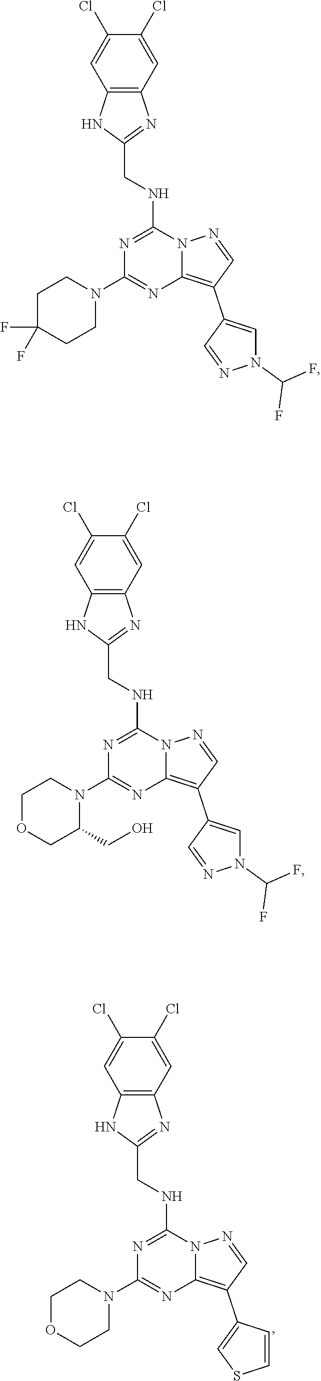 [SMILES: FC(F)n1cc(-c2cnn3c(NCc4nc5cc(Cl)c(Cl)cc5[nH]4)nc(N4CCC(F)(F)CC4)nc23)cn1 ; OC[C@@H]1COCCN1c1nc(NCc2nc3cc(Cl)c(Cl)cc3[nH]2)n2ncc(-c3cnn(C(F)F)c3)c2n1 ; Clc1cc2nc(CNc3nc(N4CCOCC4)nc4c(-c5ccsc5)cnn34)[nH]c2cc1Cl]

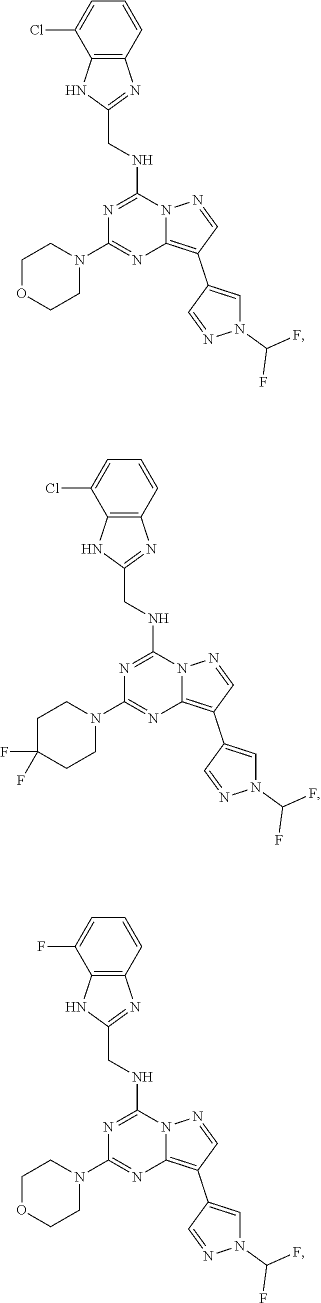 [SMILES: FC(F)n1cc(-c2cnn3c(NCc4nc5cccc(Cl)c5[nH]4)nc(N4CCOCC4)nc23)cn1 ; FC(F)n1cc(-c2cnn3c(NCc4nc5cccc(Cl)c5[nH]4)nc(N4CCC(F)(F)CC4)nc23)cn1 ; Fc1cccc2nc(CNc3nc(N4CCOCC4)nc4c(-c5cnn(C(F)F)c5)cnn34)[nH]c12]

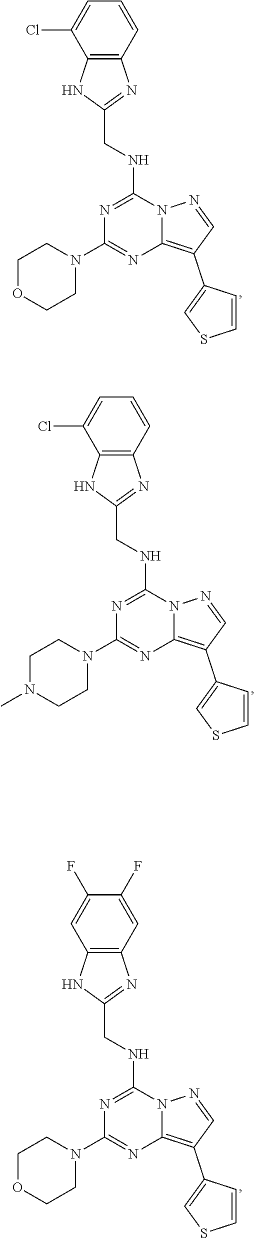 [SMILES: Clc1cccc2nc(CNc3nc(N4CCOCC4)nc4c(-c5ccsc5)cnn34)[nH]c12 ; CN1CCN(c2nc(NCc3nc4cccc(Cl)c4[nH]3)n3ncc(-c4ccsc4)c3n2)CC1 ; Fc1cc2nc(CNc3nc(N4CCOCC4)nc4c(-c5ccsc5)cnn34)[nH]c2cc1F]

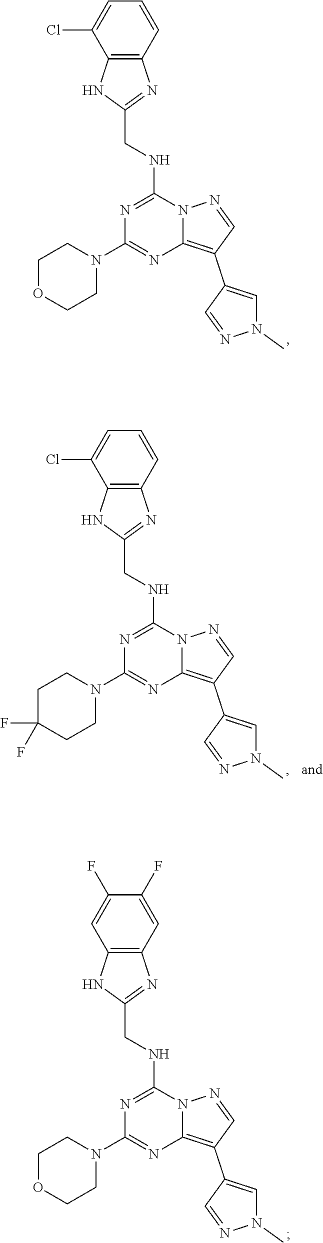 [SMILES: Cn1cc(-c2cnn3c(NCc4nc5cccc(Cl)c5[nH]4)nc(N4CCOCC4)nc23)cn1 ; Cn1cc(-c2cnn3c(NCc4nc5cccc(Cl)c5[nH]4)nc(N4CCC(F)(F)CC4)nc23)cn1 ; Cn1cc(-c2cnn3c(NCc4nc5cc(F)c(F)cc5[nH]4)nc(N4CCOCC4)nc23)cn1]

and pharmaceutically acceptable salts, solvates, hydrates, polymorphs, co-crystals, isotopically labeled derivatives, prodrugs, and compositions thereof.

Also provided are methods involving the disclosed compounds or compositions for treating and/or preventing cell proliferative diseases, including certain cancers of breast, brain, ovarian, lung, colorectal cancer, leukemia, lymphoma, melanoma, multiple myeloma, Ewing's sarcoma, osteosarcoma, and inflammatory and myotonic dystrophy type 1 diseases in a mammal.

Treatment of a subject with a proliferative disease using a compound or composition of the disclosure may inhibit the aberrant activity of kinases, such as a cyclin-dependent kinase (CDK) (e.g., CDK12/13), and therefore, induce potent antiproliferative and apoptotic effects and/or inhibit transcription in the subject.

Additional advantages will be set forth in part in the following description and in part will be obvious from the description or may be learned by practicing the aspects described below. The advantages described below will be realized and attained by the elements and combinations pointed out in the appended claims. It is to be understood that the foregoing general description and the following detailed description are exemplary and explanatory only and are not restrictive.

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, which may, of course, vary. It is also to be understood that the terminology used herein describes particular aspects only and is not intended to be limiting.

Also, throughout this specification, various publications are referenced. The disclosures of these publications in their entireties are hereby incorporated by reference into this application to more fully describe the state of the art to which the disclosed matter pertains. The references disclosed are also individually and expressly incorporated by reference herein for the material contained in them that is discussed in the sentence in which the reference is relied upon.

### General Definitions

In this specification and in the claims that follow, reference will be made to many terms, which shall be defined to have the following meanings:

Throughout the specification and claims, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context dictates otherwise. Thus, for example, reference to “a composition” includes mixtures of two or more such compositions, reference to “an inhibitor” includes mixtures of two or more such inhibitors and the like.

“Optional” or “optionally” means that the subsequently described event or circumstance can or cannot occur and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contains certain errors resulting from the standard deviation in their respective testing measurements. Furthermore, when numerical ranges of varying scope are set forth herein, it is contemplated that any combination of these values, including the recited values, may be used. Further, ranges can be expressed herein as from “about” one particular value and/or to “about” another particular value. When such a range is expressed, another aspect includes from the one particular value and/or to the other particular value. Similarly, when values are expressed as approximations, using the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

By “reduce” or other forms of the word, such as “reducing” or “reduction,” it is meant lowering of an event or characteristic (e.g., tumor growth, metastasis). It is understood that this is typically in relation to some standard or expected value. In other words, it is relative, but it is not always necessary for the standard or relative value to be referred to. For example, “reduces tumor growth” means decreasing the number of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of alleviation of one or more symptoms (such as tumor growth or metastasis), diminishment of the extent of cancer, stabilized (i.e., not worsening) state of cancer, preventing or delaying spread (e.g., metastasis) of cancer, delaying occurrence or recurrence of cancer, delay or slowing of cancer progression, amelioration of the cancer state, and remission (whether partial or total).

The term “patient” refers to a human needing treatment with an anti-cancer agent or treatment for any purpose, and more preferably, a human needing such a treatment to treat cancer or a precancerous condition or lesion. However, the term “patient” can also refer to non-human animals, preferably mammals such as dogs, cats, horses, cows, pigs, sheep, and non-human primates, among others, that need treatment with an anti-cancer agent or treatment.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight of component Y, X and Y are present at a weight ratio of 2:5 and are present in such ratio regardless of whether additional components are contained in the mixture.

Unless expressly stated to the contrary, a weight percent (wt. %) of a component is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein, which satisfy the valencies of the heteroatoms. This disclosure is not intended to be limited by the permissible substituents of organic compounds. Also, the terms “substitution” or “substituted with” include the implicit proviso that such substitution follows the permitted valence of the substituted atom and the substituent and that the substitution results in a stable compound, e.g., a compound that does not spontaneously transform such as by rearrangement, cyclization, elimination, etc.

The term “aliphatic,” as used herein, refers to a nonaromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The symbol Aⁿ is used herein as merely a generic substituent in the definitions below.

The term “alkoxy” as used herein is an alkyl group bound through a single, terminal ether linkage; that is, an “alkoxy” group can be defined as —OA¹ where A¹ is alkyl as defined above.

The term “alkenyl,” as used herein, is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl,” as used herein, is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as an aromatic group with at least one heteroatom incorporated within the ring of the aromatic group. Examples of heteroatoms include but are not limited to nitrogen, oxygen, sulfur, and phosphorus. The term “non-heteroaryl,” which is included in the term “aryl,” defines a group that contains an aromatic group that does not contain a heteroatom. The aryl and heteroaryl groups can be substituted or unsubstituted. The aryl and heteroaryl groups can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups bound together via a fused ring structure, as in naphthalene, or attached via one or more carbon-carbon bonds, as in biphenyl.

The term “cycloalkyl,” as used herein, is a nonaromatic carbon-based ring composed of at least three carbon atoms. Examples of cycloalkyl groups include but are not limited to cyclopropyl, cyclobutyl, cyclopentyl, cyclohexyl, etc. The term “heterocycloalkyl” is a cycloalkyl group defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl,” as used herein, is a nonaromatic carbon-based ring composed of at least three carbon atoms containing at least one double bond, i.e., C═C. Examples of cycloalkenyl groups include but are not limited to cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl or non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” is represented herein by the formula —C(O)H. Throughout this specification, “C(O)” is a shorthand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” is represented herein by the formula —C(O)OH. A “carboxylate” is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” refers to the halogens fluorine, chlorine, bromine, and iodine.

The term “hydroxyl” used herein is represented by the formula —OH.

The term “nitro” is represented herein by the formula —NO₂.

The term “cyano” as used herein is represented by the formula —CN

The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” is represented herein by the formula —SH.

It is to be understood that the compounds provided herein may contain chiral centers. Such chiral centers may be of the (R—) or (S—) configuration. The compounds herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to the administration of the compound in its (S—) form.

As used herein, substantially pure means sufficiently homogeneous to appear free of readily detectable impurities as determined by standard methods of analysis, such as thin-layer chromatography (TLC), nuclear magnetic resonance (NMR), gel electrophoresis, high-performance liquid chromatography (HPLC), mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. However, a substantially chemically pure compound may be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “pharmaceutically acceptable” component is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent, or vehicle, for delivering the disclosed compounds to the patient. The carrier can be liquid or solid and is selected with the planned manner of administration in mind. Liposomes are also a type of pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Except insofar as any conventional media or agent is incompatible with the active ingredient, its use in the therapeutic compositions is contemplated.

The term “therapeutically effective amount” means the amount of active compound or pharmaceutical agent that elicits the biological or medicinal response in a tissue, system, animal, or human being sought by a researcher, veterinarian, medical doctor, or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages is believed to be safe and effective.

Reference will now be made in detail to specific aspects of the disclosed materials, compounds, compositions, articles, and methods, examples of which are illustrated in the accompanying Examples.

### Compounds

The present inventors have now identified new N-((1H-benzo[d]imidazol-2-yl)methyl)pyrazolo[1,5-a][1,3,5]triazin-4-amine compounds for use in the prevention and/or treatment of proliferative diseases and conditions including cancers and myotonic dystrophy. While not wishing to be bound by theory, it is considered that these novel compounds are capable of inhibiting cell proliferation by inhibiting the activity of CDK12 and/or CDK13.

In certain aspects, disclosed herein are compounds as shown in Table 1 below.

TABLE 1
Chemical structure of compounds of the present invention.

| No. | Structure | Name | Mass |
|---|---|---|---|
| 3 | 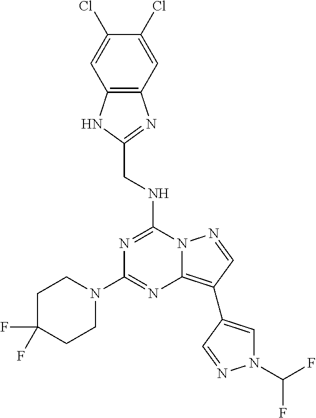 [SMILES: FC(F)n1cc(-c2cnn3c(NCc4nc5cc(Cl)c(Cl)cc5[nH]4)nc(N4CCC(F)(F)CC4)nc23)cn1] | N-((5,6-dichloro-1H- benzo[d]imidazol-2-yl)methyl)-8- (1-(difluoromethyl)-1H-pyrazol-4- yl)-2-(4,4-difluoropiperidin-1- yl)pyrazolo[1,5-a]triazin-4- amine | 568.4 |
| 4 | 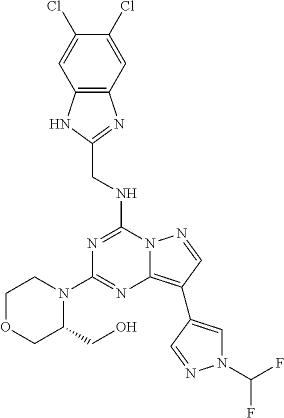 [SMILES: OC[C@@H]1COCCN1c1nc(NCc2nc3cc(Cl)c(Cl)cc3[nH]2)n2ncc(-c3cnn(C(F)F)c3)c2n1] | (R)-(4-(4-(((5,6-dichloro-1H- benzo[d]imidazol-2- yl)methyl)amino)-8-(1- (difluoromethyl)-1H-pyrazol-4- yl)pyrazolo[1,5-a][1,3,5]triazin-2- yl)morpholin-3-yl)methanol | 565.4 |
| 5 | 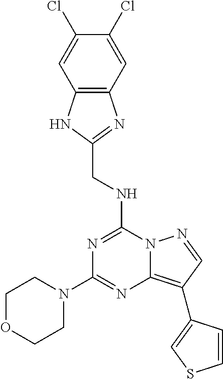 [SMILES: Clc1cc2nc(CNc3nc(N4CCOCC4)nc4c(-c5ccsc5)cnn34)[nH]c2cc1Cl] | N-((5,6-dichloro-1H- benzo[d]imidazol-2-yl)methyl)-2- morpholino-8-(thiophen-3- yl)pyrazolo[1,5-a][1,3,5]triazin-4- amine | 501.4 |
| 6 | 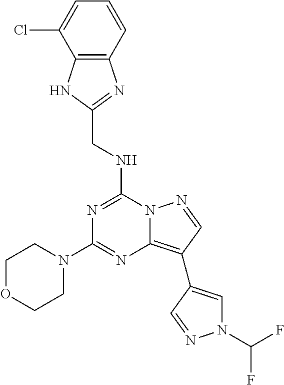 [SMILES: FC(F)n1cc(-c2cnn3c(NCc4nc5cccc(Cl)c5[nH]4)nc(N4CCOCC4)nc23)cn1] | N-((7-chloro-1H- benzo[d]imidazol-2-yl)methyl-8- (1-(difluoromethyl)-1H-pyrazol-4- yl)-2-morpholinopyrazolo[1,5- a][1,3,5]triazin-4-amine | 500.9 |
| 7 | 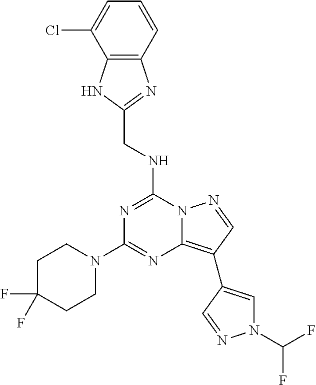 [SMILES: FC(F)n1cc(-c2cnn3c(NCc4nc5cccc(Cl)c5[nH]4)nc(N4CCC(F)(F)CC4)nc23)cn1] | N-((7-chloro-1H- benzo[d]imidazol-2-yl)methyl)-8- (1-(difluoromethyl)-1H-pyrazol-4- yl)-2-(4,4-difluoropiperidin-1- yl)pyrazolo[1,5-a][1,3,5]triazin-4- amine | 534.9 |
| 8 | 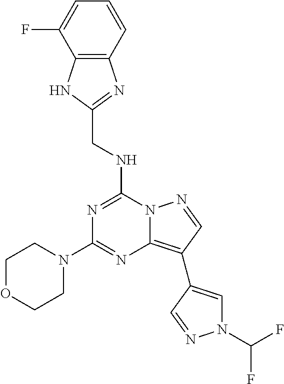 [SMILES: Fc1cccc2nc(CNc3nc(N4CCOCC4)nc4c(-c5cnn(C(F)F)c5)cnn34)[nH]c12] | 8-(1-(difluoromethyl)-1H-pyrazol- 4-yl)-N-((7-fluoro-1H- benzo[d]imidazol-2-yl)methyl)-2- morpholinopyrazolo[1,5-a][1,3,5] triazin-4-amine | 484.5 |
| 9 | 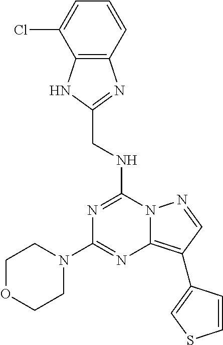 [SMILES: Clc1cccc2nc(CNc3nc(N4CCOCC4)nc4c(-c5ccsc5)cnn34)[nH]c12] | N-((7-chloro-1H- benzo[d]imidazol-2-yl)methyl)-2- morpholino-8-(thiophen-3- yl)pyrazolo[1,5-a][1,3,5]triazin-4- amine | 467 |
| 10 | 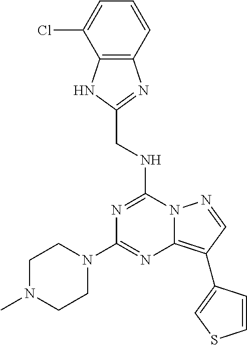 [SMILES: CN1CCN(c2nc(NCc3nc4cccc(Cl)c4[nH]3)n3ncc(-c4ccsc4)c3n2)CC1] | N-((7-chloro-1H- benzo[d]imidazol-2-yl)methyl)-2- (4-methylpiperazin-1-yl)-8- (thiophen-3-yl)pyrazolo [1,5-a][1,3,5]triazin-4-amine | 480 |
| 11 | 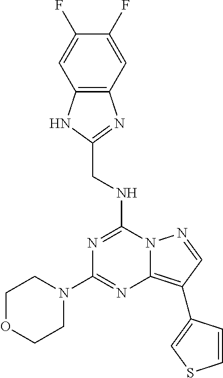 [SMILES: Fc1cc2nc(CNc3nc(N4CCOCC4)nc4c(-c5ccsc5)cnn34)[nH]c2cc1F] | N-((5,6-difluoro-1H- benzo[d]imidazol-2-yl)methyl)-2- morpholino-8-(thiophen-3- yl)pyrazolo[1,5-a][1,3,5]triazin-4- amine | 468.5 |
| 12 | 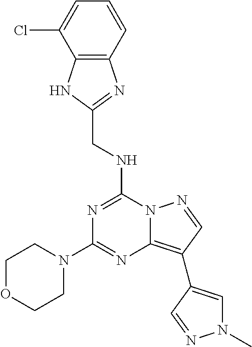 [SMILES: Cn1cc(-c2cnn3c(NCc4nc5cccc(Cl)c5[nH]4)nc(N4CCOCC4)nc23)cn1] | N-((7-chloro-1H- benzo[d]imidazol-2-yl)methyl)-8- (1-methyl-1H-pyrazol-4-yl)-2- morpholinopyrazolo [1,5-a][1,3,5]triazin-4-amine | 464.9 |
| 13 | 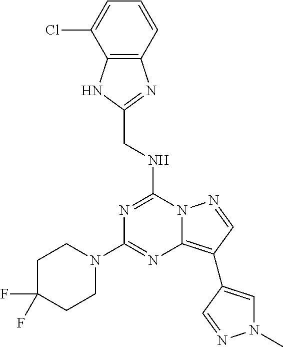 [SMILES: Cn1cc(-c2cnn3c(NCc4nc5cccc(Cl)c5[nH]4)nc(N4CCC(F)(F)CC4)nc23)cn1] | N-((7-chloro-1H- benzo[d]imidazol-2-yl)methyl)-2- (4,4-difluoropiperidin-1-yl)-8-(1- methyl-1H-pyrazol-4- yl)pyrazolo[1,5-a][1,3,5]triazin- 4-amine | 498.9 |
| 14 | 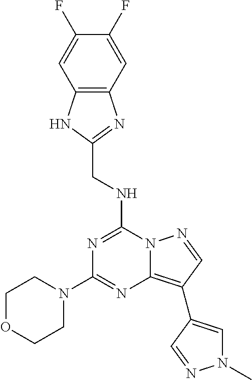 [SMILES: Cn1cc(-c2cnn3c(NCc4nc5cc(F)c(F)cc5[nH]4)nc(N4CCOCC4)nc23)cn1] | N-((5,6-difluoro-1H- benzo[d]imidazol-2-yl)methyl)-8- (1-methyl-1H-pyrazol-4-yl)-2- morpholinopyrazolo [1,5-a][1,3,5]triazin-4-amine | 466.5 |

The present disclosure further provides a method of synthesizing compounds according to the present invention, or a pharmaceutically acceptable salt, solvate, or prodrug thereof.

Concerning the description of the synthetic methods described below and in the referenced synthetic methods that are used to prepare starting materials, it will be understood by those skilled in the art that all proposed reaction conditions, including choice of solvent, reaction atmosphere, reaction temperature, duration of the experiment and workup procedures, can be readily selected. Moreover, it will be understood by those skilled in the art that the functionality present on various portions of the molecule must be compatible with the reagents and reaction conditions utilized. An example of a particularly suitable method for synthesizing a compound of the present invention is shown in Scheme 1 below.

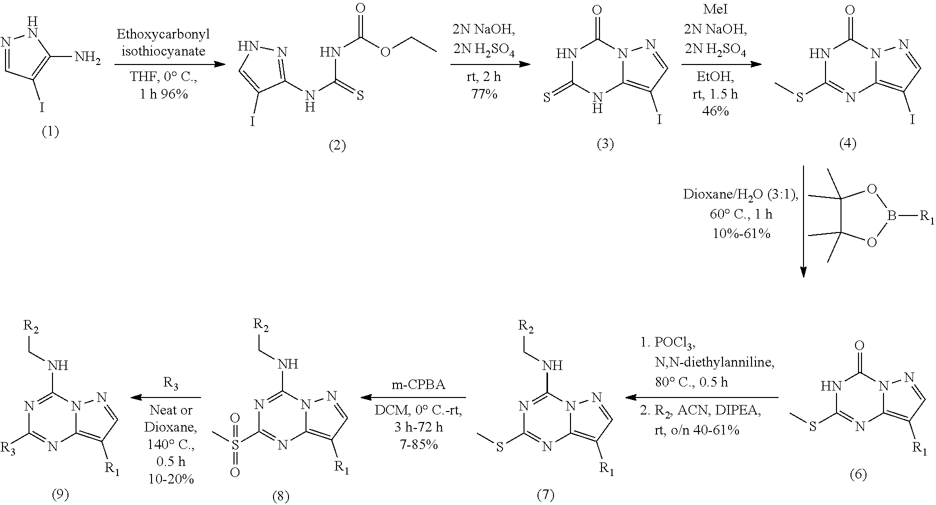

The intermediate compounds of the present disclosure may be prepared by, for example, the general synthetic methodologies described in, which is herein incorporated by reference (Journal of Medicinal Chemistry 2009, 52(3) 655-663; ChemMedChem 2019, 14(11), 1074-1078).

The present disclosure also includes compounds with at least one desired isotopic substitution of an atom at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example, ²H or ³H), detection or imaging techniques, such as positron emission tomography (PET), or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example, deuterium (²H) and tritium (³H), may be used anywhere in described structures that achieve the desired result. Alternatively or in addition, isotopes of carbon, e.g., ¹³C and ¹⁴C, may be used. In one embodiment, the isotopic substitution is replacing hydrogen with deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example, deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at the desired location. Unless otherwise stated, the enrichment at any point is above natural abundance and, in an embodiment, is enough to alter a detectable property of the compounds as a drug in a human.

The compounds of the present disclosure may form a solvate with solvents (including water). Therefore, the invention includes a solvated form of the active compound in one embodiment. The term “solvate” refers to a molecular complex of a compound of the present invention (including a salt thereof) with one or more solvent molecules. Non-limiting examples of solvents are water, ethanol, dimethyl sulfoxide, acetone, and other common organic solvents. The term “hydrate” refers to a molecular complex comprising a disclosed compound and water. Pharmaceutically acceptable solvates per the invention include those wherein the solvent of crystallization may be isotopically substituted, e.g., D₂O, d₆-acetone, or d₆-DMSO. A solvate can be in a liquid or solid form.

A “prodrug” as used herein means a compound that, when administered to a host in vivo, is converted into a parent drug. As used herein, the term “parent drug” means any presently described compounds herein. Prodrugs can be used to achieve any desired effect, including enhancing the properties of the parent drug or improving the pharmaceutic or pharmacokinetic properties of the parent, including increasing the drug's half-life in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain embodiments, the prodrug renders the parent compound more lipophilic. In certain embodiments, a prodrug can be provided with several prodrug moieties in a linear, branched, or cyclic manner. For example, non-limiting embodiments include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety and are typically biodegradable in vivo. In some embodiments, 2, 3, 4, or 5 biodegradable prodrug moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some embodiments, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched, or cyclic) to one or more other prodrug moieties to modify the parent drug to achieve the desired performance, such as increased half-life, lipophilicity, or additional drug delivery or pharmacokinetic properties. The amino acid can be any compound with an amino group and a carboxylic acid, including an aliphatic amino acid, alkyl amino acid, aromatic amino acid, heteroaliphatic amino acid, heteroalkyl amino acid, heterocyclic amino acid, or heteroaryl amino acid.

A pharmaceutical composition comprising a therapeutically effective amount of a compound described herein, or a pharmaceutically acceptable salt thereof, and a pharmaceutically acceptable carrier are also described.

### METHODS

Further provided herein are methods of inhibiting a cyclin-dependent kinase, comprising contacting the cyclin dependent-kinase with an effective amount or concentration of a compound or pharmaceutical composition as disclosed herein. The cyclin-dependent kinase can be a cyclin-dependent kinase12 or 13 (CDK12 or CDK13). In some aspects of the methods disclosed herein, the CDK is disposed within the body tissue of a patient with cancer, inflammatory or myotonic dystrophy type 1 diseases, or a combination thereof.

Further provided herein are methods for the treatment of a disorder of uncontrolled cellular proliferation in a mammal comprising the step of administering to the mammal an effective amount of a compound as disclosed herein. In some examples, the disorder can be cancer. The methods can further comprise administering a second compound or composition, such as, for example, anti-cancer agents or anti-inflammatory agents. Additionally, the method can further comprise administering an effective amount of ionizing radiation to the subject.

Methods of killing a tumor cell are also provided herein. The methods comprise contacting a tumor cell with an effective amount of a compound or composition as disclosed herein. The methods can further include administering a second compound or composition (e.g., an anti-cancer agent or an anti-inflammatory agent) or administering an effective amount of ionizing radiation to the subject.

Also provided herein are methods of radiotherapy of tumors, comprising contacting the tumor with an effective amount of a compound or composition as disclosed herein and irradiating the tumor with an effective amount of ionizing radiation.

Also disclosed are methods for treating oncological disorders in a patient. In one embodiment, an effective amount of one or more compounds or compositions disclosed herein is administered to a patient having an oncological disorder and who is in need of treatment thereof. The disclosed methods can optionally include identifying a patient who is or can be in need of treatment of an oncological disorder. The patient can be a human or other mammals, such as a primate (monkey, chimpanzee, ape, etc.), dog, cat, cow, pig, or horse, or other animals having an oncological disorder. Oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Kaposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

In some examples, the cancer is selected from breast cancer, brain cancer, cervical cancer, chronic myeloproliferative disorder, colorectal cancer, Ewing's sarcoma, gastrointestinal cancer, glioma, leukemia, lung cancer, lymphoma, endometrial cancer, melanoma, multiple myeloma, myelodysplastic syndrome, myeloproliferative neoplasm, pancreatic cancer, plasma cell neoplasm (myeloma), prostate cancer, ovarian cancer, osteosarcoma, skin cancer, testicular cancer, and thyroid cancer.

In some aspects, disclosed are methods for treating a tumor or tumor metastases in a subject by the administration to the subject a combination of at least one compound or composition as disclosed herein and at least one cancer immunotherapeutic agent. The disclosed compounds can be administered alone or in combination with a cancer immunotherapeutic agent. The subject can receive the therapeutic compositions before, during, or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

In specific examples, the type of cancer is breast cancer or lung cancer.

Methods of treating a disease selected from inflammatory or myotonic dystrophy type diseases in a mammal comprising the step of administering to the mammal an effective amount of a compound as described herein are provided. In some aspects, the mammal is diagnosed with inflammatory or myotonic dystrophy type 1 disease.

### Administration

The disclosed compounds can be administered sequentially or simultaneously in separate or combined pharmaceutical formulations. When one or more of the disclosed compounds is combined with a second therapeutic agent, the dose of each compound can be either the same or differ from that when the compound is used alone. Appropriate doses will be readily appreciated by those skilled in the art.

The term “administration” and variants thereof (e.g., “administering” a compound) in reference to a compound of the invention means introducing the compound or a prodrug of the compound into the system of the animal in need of treatment. When a compound of the invention or prodrug thereof is combined with one or more other active agents (e.g., a cytotoxic agent, etc.), “administration” and its variants are each understood to include the concurrent and sequential introduction of the compound or prodrug thereof and other agents.

In vivo application of the disclosed compounds and compositions containing them can be accomplished by any suitable method and technique presently or prospectively known to those skilled in the art. For example, the disclosed compounds can be formulated in a physiologically- or pharmaceutically-acceptable form and administered by any suitable route known in the art, including oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration or at continuous or distinct intervals as readily determined by a person skilled in the art.

The compounds disclosed herein and compositions comprising them can also be administered utilizing liposome technology, slow-release capsules, implantable pumps, and biodegradable containers. These delivery methods can provide a uniform dosage over an extended period. The compounds can also be administered in their salt derivative forms or crystalline forms.

The compounds disclosed herein can be formulated according to known methods for preparing pharmaceutically acceptable compositions. Formulations are described in detail in many sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier to facilitate the effective administration of the compound. The compositions used can also be in a variety of forms. These include, for example, solid, semisolid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example, sealed ampoules and vials. The formulations can be stored in a freeze-dried (lyophilized) condition requiring only the condition of the sterile liquid carrier, for example, water for injections, before use. Extemporaneous injection solutions and suspensions can be prepared from sterile powder, granules, tablets, etc. It should be understood that in addition to the ingredients particularly mentioned above, the compositions disclosed herein can include other agents conventional in the art regarding the type of formulation in question.

Compounds disclosed herein and compositions comprising them can be delivered to a cell either through direct contact with the cell or via a carrier. Carriers for delivering compounds and compositions to cells are known in the art and include, for example, encapsulating the composition in a liposome moiety. Another means for delivering compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition, allowing the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anti-cancer substances and/or with radiation and/or photodynamic therapy and/or with surgical treatment to remove a tumor. These other substances or treatments can be given simultaneously or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have a viral genome present in the tumor or cancer cells. For example, Epstein-Barr Virus (EBV) is associated with several mammalian malignancies. The compounds disclosed herein can also be used alone or in combination with anti-cancer or antiviral agents, such as ganciclovir, azidothymidine (AZT), lamivudine (3TC), etc., to treat patients infected with a virus that can cause cellular transformation and/or treat patients having a tumor or cancer that is associated with the presence of viral genome in the cells. The compounds disclosed herein can also be used in combination with viral-based treatments of oncologic disease. For example, the compounds can be used with mutant herpes simplex virus in the treatment of non-small cell lung cancer (Toyoizumi et al., “Combined therapy with chemotherapeutic agents and herpes simplex virus type IICP34.5 mutant (HSV-1716) in human non-small cell lung cancer,” Human Gene Therapy, 1999, 10(18):17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent or an assimilable edible carrier for oral delivery. They can be enclosed in hard or soft shell gelatin capsules, compressed into tablets, or incorporated directly with the food of the patient's diet. For oral therapeutic administration, the active compound can be combined with one or more excipients and used as ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch, or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to modify the solid unit dosage form's physical form. For instance, tablets, pills, or capsules can be coated with gelatin, wax, shellac, sugar, and the like. A syrup or elixir can contain the active compound, sucrose or fructose as a sweetening agent, methyl and propylparabens as preservatives, a dye, and flavorings such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a non-toxic surfactant. Dispersions can also be prepared in glycerol, liquid polyethylene glycols, triacetin, oils, and mixtures thereof. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, non-toxic glyceryl esters, and suitable mixtures thereof. The proper fluidity can be maintained, for example, by the formation of liposomes, by the maintenance of the required particle size in the case of dispersions, or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by including agents that delay absorption, such as aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

For topical administration, compounds and agents disclosed herein can be applied as a liquid or solid. However, it will generally be desirable to administer them topically to the skin as compositions, combined with a dermatologically acceptable carrier, which can be a solid or a liquid. Compounds, agents, and compositions disclosed herein can be applied topically to a subject's skin to reduce the size (and can include complete removal) of malignant or benign growths or treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for the delivery of pharmacological substances to dermal lesions can also be used, such as that described in U.S. Pat. No. 5,167,649.

Useful solid carriers include finely divided solids such as talc, clay, microcrystalline cellulose, silica, alumina, and the like. Useful liquid carriers include water, alcohols or glycols, or water-alcohol/glycol blends, where the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical, or parenteral administration, comprising an amount of a compound, constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors, including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment before, after, or in combination with other antitumor or anti-cancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same or different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish), etc.) Also disclosed are methods for treating an oncological disorder comprising administering an effective amount of a compound and/or agent disclosed herein before, after, and/or in combination with administration of a chemotherapeutic agent, an immunotherapeutic agent, a radiotherapeutic agent, or radiotherapy.

### Kits

Kits for practicing the methods of the invention are further provided. By “kit” is intended any manufacture (e.g., a package or a container) comprising at least one reagent, e.g., any one of the compounds described in Table 1. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those described herein.

In one embodiment, a kit includes instructions or packaging materials that describe how to administer a compound or composition of the kit. Containers of the kit can be of any suitable material, e.g., glass, plastic, metal, etc., and any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

The following examples are set forth below to illustrate the methods and results according to the disclosed subject matter. These examples are not intended to include all aspects of the subject matter disclosed herein but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric. Numerous variations and combinations of reaction conditions, e.g., component concentrations, temperatures, pressures, and other reaction ranges and conditions, can be used to optimize the product purity and yield obtained from the described process. Only reasonable and routine experimentation will be required to optimize such process conditions.

### Example 1: Synthesis of CDK12/13 Inhibitors

General. All reagents were purchased from commercial suppliers and were used without further purification. Dichloromethane, diethyl ether, N,N-dimethylformamide and tetrahydrofuran were dried by being passed through a column of desiccant (activated A-1 alumina). Triethylamine and diisopropyl amine were purified by distillation from calcium hydride. Reactions were either monitored by thin layer chromatography or analytical LC-MS. Thin layer chromatography was performed on Kieselgel 60 F254 glass plates pre-coated with a 0.25 mm thickness of silica gel. TLC plates were visualized with UV light and/or by staining with ninhydrin solution. Normal phase column chromatography was performed on a Biotage Selekt automated flash system. Compounds were loaded onto pre-filled cartridges filled with KP-Sil 50 μm irregular silica. For microwave reactions, a Biotage Initiator Microwave system was used. Some of the final products were isolated by reverse-phase HPLC using Waters HPLC system with UV detector, with Atlantis T3 OBD Prep Column, 100 Å, 5 μm, 19 mm×150 mm. Compounds were eluted using a gradient elution of 90/10 to 0/100 A/B over 20 min at a flow rate of 20.0 mL/min, where solvent A was water (+0.1% ammonium acetate) and solvent B was acetonitrile.

The structures of all compounds were verified via ¹H NMR, ¹³C NMR, ¹⁹F NMR and LCMS. The purity of isolated products was determined using an LC-MS instrument (Agilent 1290 Infinity series LC with single quadrupole MSD system, AP-ESI Ion Source) equipped with Kinetex® 1.7 μm C18 100 Å, LC Column 50×2.1 mm, Ea (Phenomenex) column. Elution was performed using the following conditions: 2% (v/v) acetonitrile (+0.1% FA) in 98% (v/v) H₂O (+0.1% FA), ramped to 98% acetonitrile over 4.0 min, and holding at 98% acetonitrile for 0.5 min with a flow rate of 0.6 mL/min; UV absorption was detected from 200 to 950 nm using a diode array detector. The purity of each compound was ≥95% based on this analysis.

NMR spectra were recorded at ambient temperature on a 500 MHz Bruker NMR spectrometer in DMSO-d6. All ¹H NMR data are reported in parts per million (ppm) downfield of TMS and were measured relative to the signals for dimethyl sulfoxide (2.50 ppm). All ¹³C NMR spectra are reported in ppm relative to the signals for dimethyl sulfoxide (39.5 ppm) with ¹H decoupled observation. ¹⁹F NMR experiments were performed with ¹H decoupling. Data for ¹H NMR are reported as follows: chemical shift (δ, ppm), multiplicity (s=singlet, d=doublet, t=triplet, q=quartet, m=multiplet), integration, and coupling constant (Hz), whereas ¹³C NMR analyses were obtained at 125 Mhz and reported in terms of chemical shift. NMR data was analyzed and processed by using MestReNova software.

### N-((5,6-dichloro-1H-benzo[d]imidazol-2-yl)methyl)-8-(1-methyl-1H-pyrazol-4-yl)-2-morpholinopyrazolo[1,5-a][1,3,5]triazin-4-amine (1)

¹H NMR (500 MHz, DMSO) δ 12.65 (s, 1H), 9.02 (s, 1H), 8.20 (s, 1H), 7.99 (s, 1H), 7.82 (s, 1H), 7.70 (s, 1H), 6.62 (s, 1H), 4.87 (d, J=4.1 Hz, 2H), 3.86 (s, 3H), 3.64 (s, 4H), 3.53 (s, 4H).

### N-((5,6-dichloro-1H-benzo[d]imidazol-2-yl)methyl)-8-(1-(difluoromethyl)-1H-pyrazol-4-yl)-2-morpholinopyrazolo[1,5-a][1,3,5]triazin-4-amine (2)

¹H NMR (500 MHz, DMSO) δ 9.87 (s, 1H), 9.50 (s, 1H), 9.10 (s, 1H), 8.46 (s, 1H), 8.34 (s, 1H), 8.25 (s, 1H), 7.84 (s, 1H), 7.76 (s, 1H), 4.88 (s, 2H), 3.66 (s, 4H), 3.53 (s, 4H).

### N-((5,6-dichloro-1H-benzo[d]imidazol-2-yl)methyl)-8-(1-(difluoromethyl)-1H-pyrazol-4-yl)-2-(4,4-difluoropiperidin-1-yl)pyrazolo[1,5-a][1,3,5]triazin-4-amine (3)

¹H NMR (500 MHz, DMSO) δ 8.47 (s, 1H), 8.35 (s, 1H), 8.30 (s, 1H), 8.26 (s, 1H), 7.98 (s, 1H), 7.84 (s, 1H), 7.73 (s, 2H), 4.87 (s, 2H), 3.82 (s, 4H), 1.80 (s, 4H).

### (R)-(4-(4-(((5,6-dichloro-1H-benzo[d]imidazol-2-yl)methyl)amino)-8-(1-(difluoromethyl)-1H-pyrazol-4-yl)pyrazolo[1,5-a][1,3,5]triazin-2-yl)morpholin-3-yl)methanol (4)

¹H NMR (500 MHz, DMSO) δ 8.45 (s, 1H), 8.31 (s, 1H), 8.26 (s, 1H), 7.96 (s, 1H), 7.84 (s, 1H), 7.77-7.70 (m, 1H), 7.67 (s, 2H), 6.28 (s, 2H), 4.87 (d, J=35.6 Hz, 1H), 4.53 (s, 1H), 3.17 (s, 8H).

### N-((5,6-dichloro-1H-benzo[d]imidazol-2-yl)methyl)-2-morpholino-8-(thiophen-3-yl)pyrazolo[1,5-a][1,3,5]triazin-4-amine (5)

¹H NMR (500 MHz, DMSO) δ 8.38 (s, 1H), 7.79 (d, J=2.5 Hz, 1H), 7.75-7.68 (m, 3H), 7.56 (dd, J=5.0, 2.9 Hz, 1H), 4.87 (s, 2), 3.69-3.65 (m, 4H), 3.54 (s, 4H).

### N-((7-chloro-1H-benzo[d]imidazol-2-yl)methyl)-8-(1-(difluoromethyl)-1H-pyrazol-4-yl)-2-morpholinopyrazolo[1,5-a][1,3,5]triazin-4-amine (6)

¹H NMR (500 MHz, DMSO) δ 12.63 (s, 1H), 9.11 (s, 1H), 8.47 (s, 1H), 8.35 (s, 1H), 8.26 (s, 1H), 7.85 (t, J=59.3 Hz, 1H), 7.44-7.39 (m, 1H), 7.23 (d, J=7.7 Hz, 1H), 7.15 (t, J=7.8 Hz, 1H), 4.92 (s, 2H), 3.73-3.65 (m, 4H), 3.53 (s, 4H).

### N-((7-chloro-1H-benzo[d]imidazol-2-yl)methyl)-8-(1-(difluoromethyl)-1H-pyrazol-4-yl)-2-(4,4-difluoropiperidin-1-yl)pyrazolo[1,5-a][1,3,5]triazin-4-amine (7)

¹H NMR (500 MHz, DMSO) δ 12.60 (m, 1H), 9.18 (s, 1H), 8.48 (s, 1H), 8.37 (s, 1H), 8.27 (s, 1H), 7.86 (t, J=59.3 Hz, 1H), 7.40 (d, J=8.0 Hz, 1H), 7.23 (d, J=7.7 Hz, 1H), 7.15 (t, J=7.9 Hz, 1H), 4.92 (s, 2H), 3.83 (s, 4H), 1.82 (s, 4H).

### 8-(1-(difluoromethyl)-1H-pyrazol-4-yl)-N-((7-fluoro-1H-benzo[d]imidazol-2-yl)methyl)-2-morpholinopyrazolo[1,5-a][1,3,5]triazin-4-amine (8)

¹H NMR (500 MHz, DMSO) δ 12.61 (s, 1H), 9.08 (s, 1H), 8.47 (s, 1H), 8.34 (s, 1H), 8.26 (s, 1H), 7.84 (s, 1H), 7.28 (d, J=8.1 Hz, 1H), 7.12 (td, J=8.0, 4.8 Hz, 1H), 6.95 (dd, J=11.1, 7.9 Hz, 1H), 4.90 (s, 2H), 3.68 (s, 4H), 3.48-3.51 (m, 4H).

### N-((7-chloro-1H-benzo[d]imidazol-2-yl)methyl)-2-morpholino-8-(thiophen-3-yl)pyrazolo[1,5-a][1,3,5]triazin-4-amine (9)

¹H NMR (500 MHz, DMSO) δ 8.38 (s, 1H), 7.79 (dd, J=3.0, 1.2 Hz, 1H), 7.70 (dd, J=5.0, 1.2 Hz, 1H), 7.56 (dd, J=5.0, 3.0 Hz, 1H), 7.42 (d, J=8.0 Hz, 1H), 7.24-7.15 (m, 1H), 7.13 (t, J=7.9 Hz, 1H), 4.91 (s, 2H), 3.50-3.47 (m, 4H), 2.75-2.57 (m, 4H).

### N-((7-chloro-1H-benzo[d]imidazol-2-yl)methyl)-2-(4-methylpiperazin-1-yl)-8-(thiophen-3-yl)pyrazolo[1,5-a][1,3,5]triazin-4-amine (10)

¹H NMR (500 MHz, DMSO) δ 12.64 (s, 1H), 8.98 (s, 1H), 8.30 (s, 1H), 7.73-7.67 (m, 1H), 7.63 (dd, J=5.1, 1.2 Hz, 1H), 7.49 (dd, J=5.0, 3.0 Hz, 1H), 7.35 (d, J=7.9 Hz, 1H), 7.14 (d, J=7.6 Hz, 1H), 7.07 (t, J=7.9 Hz, 1H), 5.69 (s, 3H), 4.83 (s, 2H), 3.61 (s, 2H), 3.52 (s, 2H), 2.06 (s, 4H).

### N-((7-chloro-1H-benzo[d]imidazol-2-yl)methyl)-8-(1-methyl-1H-pyrazol-4-yl)-2-morpholinopyrazolo[1,5-a][1,3,5]triazin-4-amine (12)

¹H NMR (500 MHz, DMSO) δ 12.62 (s, 1H), 9.05 (s, 1H), 8.21 (s, 1H), 8.00 (s, 1H), 7.83 (d, J=0.8 Hz, 1H), 7.40 (d, J=7.8 Hz, 1H), 7.22 (d, J=7.7 Hz, 1H), 7.15 (t, J=7.9 Hz, 1H), 4.91 (s, 2H), 3.87 (s, 4H), 3.69-3.64 (m, 4H), 3.53 (s, 3H).

### N-((7-chloro-1H-benzo[d]imidazol-2-yl)methyl)-2-(4,4-difluoropiperidin-1-yl)-8-(1-methyl-1H-pyrazol-4-yl)pyrazolo[1,5-a][1,3,5]triazin-4-amine (13)

¹H NMR (500 MHz, DMSO) δ 12.62 (s, 1H), 9.11 (s, 1H), 8.23 (s, 1H), 8.00 (s, 1H), 7.84 (d, J=0.7 Hz, 1H), 7.40 (s, 1H), 7.23 (d, J=7.7 Hz, 1H), 7.15 (t, J=7.9 Hz, 1H), 4.90 (s, 2H), 3.87 (s, 3H), 3.81 (s, 4H), 1.80 (s, 4H).

### Example 2. Biological Activity of CDK12/13 Inhibitors

The inhibitory and antiproliferative activities of selected compounds disclosed herein against cyclin-dependent kinase 12 were investigated. Table 2 provides a summary of these activities.

TABLE 2
Inhibition of cyclin-dependent kinase 12 and antiproliferative
activity of selected compounds of the present invention.

| | CDK12 inhibition, | MDA-MB-231, |
|---|---|---|
| Compound | IC₅₀ (nM) | GI₅₀ (nM) |
| 1 | 554 | 419 |
| 2 | 262 | 150 |
| 3 | >10,000 | 710 |
| 4 | 4050 | 384 |
| 5 | 2420 | 764 |
| 6 | 65 | 62 |
| 7 | 552 | 980 |
| 8 | 44 | 44 |
| 9 | 377 | 227 |
| 10 | 1560 | 835 |
| 11 | 196 | 61 |
| 12 | 289 | 1108 |
| 13 | 1280 | 3771 |
| 14 | 1350 | 1244 |

Summary: The present disclosure relates to a novel class of inhibitors of protein kinases, in particular members of the cyclin dependent kinases, useful in the treatment of proliferative cell diseases and conditions including cancers, inflammatory and myotonic dystrophy type 1 diseases.

Advantages: Different human cancers involve deregulation of transcription-CDKs processes. CDK9 was considered the only transcription-CDK with a causative role in cancer until recently. New evidence supports the importance of CDK12 in transcription and RNA processing, maintaining genomic stability/integrity and tumorigenesis. This chemical series offers new opportunities for treatment of cancers with CDK12 mutations and defective DNA repair.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A compound selected from: 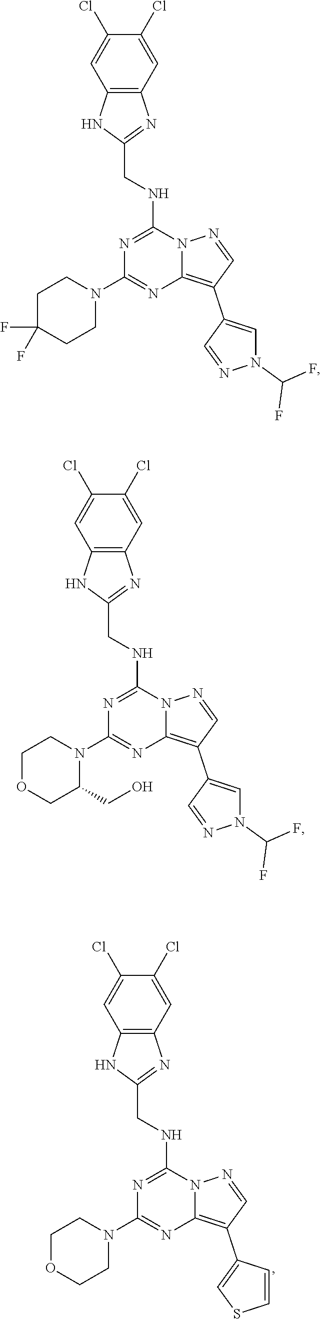 [SMILES: FC(F)n1cc(-c2cnn3c(NCc4nc5cc(Cl)c(Cl)cc5[nH]4)nc(N4CCC(F)(F)CC4)nc23)cn1 ; OC[C@@H]1COCCN1c1nc(NCc2nc3cc(Cl)c(Cl)cc3[nH]2)n2ncc(-c3cnn(C(F)F)c3)c2n1 ; Clc1cc2nc(CNc3nc(N4CCOCC4)nc4c(-c5ccsc5)cnn34)[nH]c2cc1Cl] 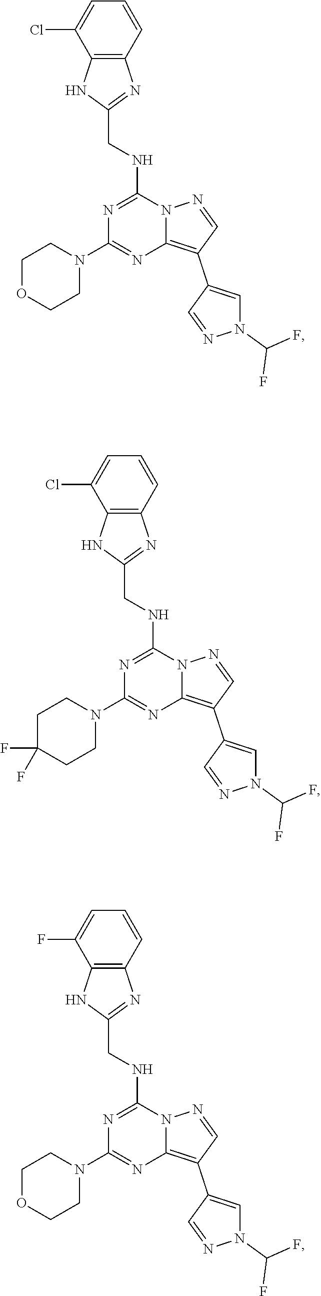 [SMILES: FC(F)n1cc(-c2cnn3c(NCc4nc5cccc(Cl)c5[nH]4)nc(N4CCOCC4)nc23)cn1 ; FC(F)n1cc(-c2cnn3c(NCc4nc5cccc(Cl)c5[nH]4)nc(N4CCC(F)(F)CC4)nc23)cn1 ; Fc1cccc2nc(CNc3nc(N4CCOCC4)nc4c(-c5cnn(C(F)F)c5)cnn34)[nH]c12] 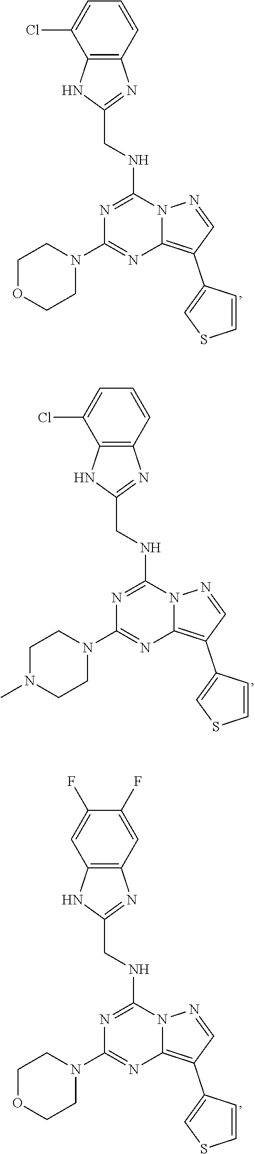 [SMILES: Clc1cccc2nc(CNc3nc(N4CCOCC4)nc4c(-c5ccsc5)cnn34)[nH]c12 ; CN1CCN(c2nc(NCc3nc4cccc(Cl)c4[nH]3)n3ncc(-c4ccsc4)c3n2)CC1 ; Fc1cc2nc(CNc3nc(N4CCOCC4)nc4c(-c5ccsc5)cnn34)[nH]c2cc1F] 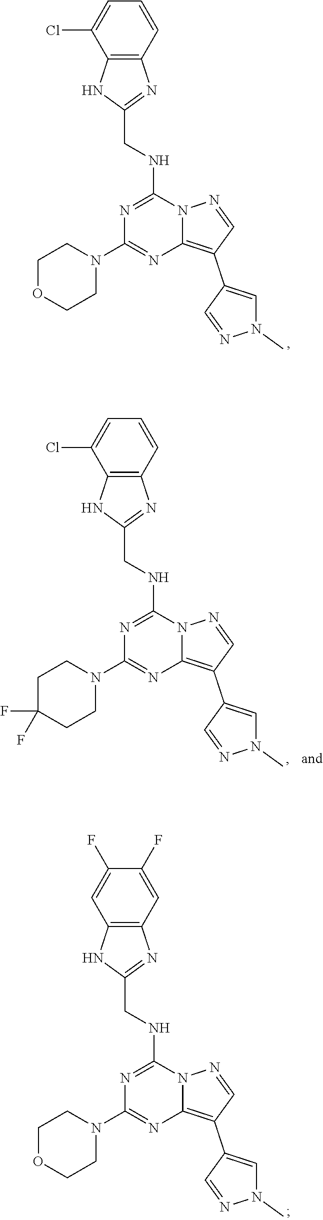 [SMILES: Cn1cc(-c2cnn3c(NCc4nc5cccc(Cl)c5[nH]4)nc(N4CCOCC4)nc23)cn1 ; Cn1cc(-c2cnn3c(NCc4nc5cccc(Cl)c5[nH]4)nc(N4CCC(F)(F)CC4)nc23)cn1 ; Cn1cc(-c2cnn3c(NCc4nc5cc(F)c(F)cc5[nH]4)nc(N4CCOCC4)nc23)cn1]
or a pharmaceutically acceptable salt thereof.

2. The compound of claim 1, wherein the compound is 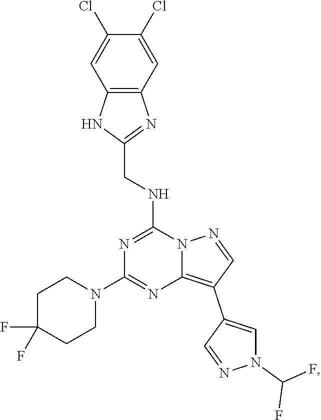 [SMILES: FC(F)n1cc(-c2cnn3c(NCc4nc5cc(Cl)c(Cl)cc5[nH]4)nc(N4CCC(F)(F)CC4)nc23)cn1]
or a pharmaceutically acceptable salt thereof.

3. The compound of claim 1, wherein the compound is 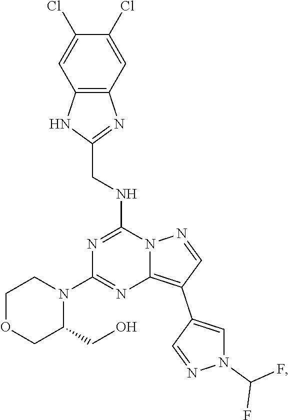 [SMILES: OC[C@@H]1COCCN1c1nc(NCc2nc3cc(Cl)c(Cl)cc3[nH]2)n2ncc(-c3cnn(C(F)F)c3)c2n1]
or a pharmaceutically acceptable salt thereof.

4. The compound of claim 1, wherein the compound is 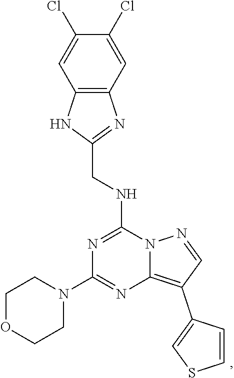 [SMILES: Clc1cc2nc(CNc3nc(N4CCOCC4)nc4c(-c5ccsc5)cnn34)[nH]c2cc1Cl]
or a pharmaceutically acceptable salt thereof.

5. The compound of claim 1, wherein the compound is 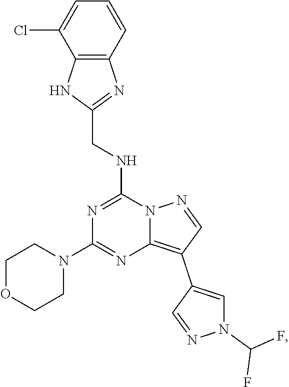 [SMILES: FC(F)n1cc(-c2cnn3c(NCc4nc5cccc(Cl)c5[nH]4)nc(N4CCOCC4)nc23)cn1]
or a pharmaceutically acceptable salt thereof.

6. The compound of claim 1, wherein the compound is 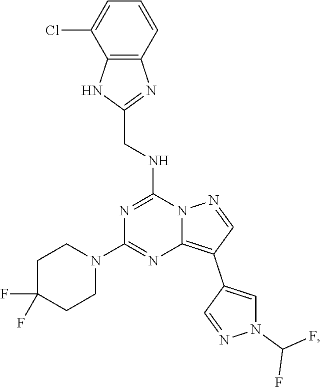 [SMILES: FC(F)n1cc(-c2cnn3c(NCc4nc5cccc(Cl)c5[nH]4)nc(N4CCC(F)(F)CC4)nc23)cn1]
or a pharmaceutically acceptable salt thereof.

7. The compound of claim 1, wherein the compound is 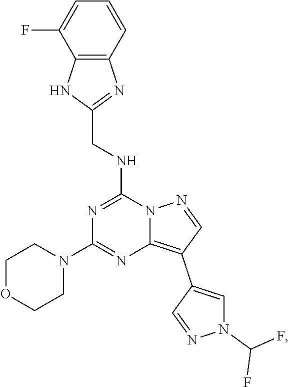 [SMILES: Fc1cccc2nc(CNc3nc(N4CCOCC4)nc4c(-c5cnn(C(F)F)c5)cnn34)[nH]c12]
or a pharmaceutically acceptable salt thereof.

8. The compound of claim 1, wherein the compound is 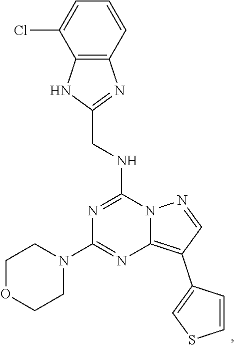 [SMILES: Clc1cccc2nc(CNc3nc(N4CCOCC4)nc4c(-c5ccsc5)cnn34)[nH]c12]
or a pharmaceutically acceptable salt thereof.

9. The compound of claim 1, wherein the compound is 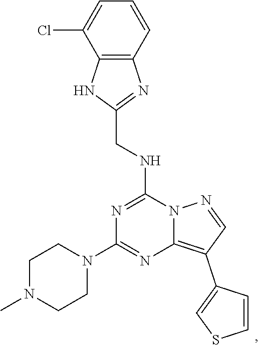 [SMILES: CN1CCN(c2nc(NCc3nc4cccc(Cl)c4[nH]3)n3ncc(-c4ccsc4)c3n2)CC1]
or a pharmaceutically acceptable salt thereof.

10. The compound of claim 1, wherein the compound is 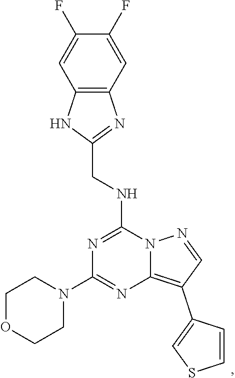 [SMILES: Fc1cc2nc(CNc3nc(N4CCOCC4)nc4c(-c5ccsc5)cnn34)[nH]c2cc1F]
or a pharmaceutically acceptable salt thereof.

11. The compound of claim 1, wherein the compound is 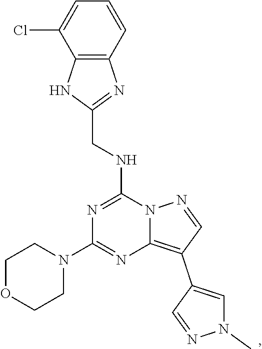 [SMILES: Cn1cc(-c2cnn3c(NCc4nc5cccc(Cl)c5[nH]4)nc(N4CCOCC4)nc23)cn1]
or a pharmaceutically acceptable salt thereof.

12. The compound of claim 1, wherein the compound is 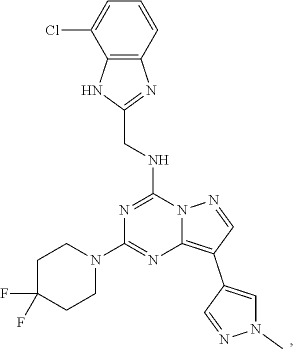 [SMILES: Cn1cc(-c2cnn3c(NCc4nc5cccc(Cl)c5[nH]4)nc(N4CCC(F)(F)CC4)nc23)cn1]
or a pharmaceutically acceptable salt thereof.

13. The compound of claim 1, wherein the compound is 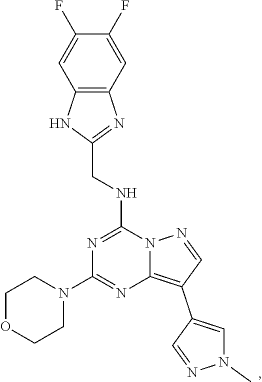 [SMILES: Cn1cc(-c2cnn3c(NCc4nc5cc(F)c(F)cc5[nH]4)nc(N4CCOCC4)nc23)cn1]
or a pharmaceutically acceptable salt thereof.

14. A pharmaceutical composition comprising a therapeutically effective amount of a compound of claim 1, or a pharmaceutically acceptable salt thereof, and a pharmaceutically acceptable carrier.

15. A method of inhibiting a cyclin-dependent kinase, comprising contacting the cyclin dependent-kinase with an effective amount or concentration of a compound of claim 1, or a pharmaceutically acceptable salt thereof.

16. The method of claim 15, wherein the cyclin-dependent kinase is cyclin-dependent kinase 12 or 13 (CDK12 or CDK13).

17. The method of claim 15, wherein the CDK is disposed within the body tissue of a patient afflicted with cancer, inflammatory or myotonic dystrophy type 1 diseases, or a combination thereof.

18. A method of treating a disorder of uncontrolled cellular proliferation in a mammal comprising administering to the mammal an effective amount of a compound of claim 1, or a pharmaceutically acceptable salt thereof.

19. (canceled)

20. (canceled)

21. The method of claim 18, wherein the disorder is cancer.

22. The method of claim 21, wherein the cancer is selected from breast cancer, brain cancer, cervical cancer, chronic myeloproliferative disorder, colorectal cancer, Ewing's sarcoma, gastrointestinal cancer, glioma, leukemia, lung cancer, lymphoma, endometrial cancer, melanoma, multiple myeloma, myelodysplastic syndrome, myeloproliferative neoplasm, pancreatic cancer, plasma cell neoplasm (myeloma), prostate cancer, ovarian cancer, osteosarcoma, skin cancer, testicular cancer, and thyroid cancer.

23. A method for the treatment of a disease selected from inflammatory or myotonic dystrophy type diseases in a mammal comprising administering to the mammal an effective amount of a compound of claim 1, or a pharmaceutically acceptable salt thereof.

24. (canceled)

25. The method of claim 23, wherein the mammal is diagnosed with inflammatory or myotonic dystrophy type 1 disease.
